(19) United States
(12) Patent Application Publication
(10) Pub. No.: US 2016/0038590 A1
(43) Pub. Date: Feb. 11, 2016

(54) COMPOSITIONS AND METHODS FOR DELIVERING THERAPEUTIC AND IMAGING AGENTS TO THE SINUSES AND MIDDLE EAR

(71) Applicants: Jian-Dong LI, Marietta, GA (US); GEORGIA STATE UNIVERSITY RESEARCH FOUNDATION, INC., Atlanta, GA (US)

(72) Inventor: Jian-Dong Li, Marietta, GA (US)

(21) Appl. No.: 14/777,173

(22) PCT Filed: Mar. 15, 2014
(86) PCT No.: PCT/US14/30046

§ 371 Date: Sep. 15, 2015

Related U.S. Application Data
(60) Provisional application No. 61/800,654, filed on Mar. 15, 2013.

(51) Int. Cl. A61K 41/00; A61K 47/48; C12N 15/113; A61K 9/00

(52) U.S. Cl. CPC A61K 41/00; A61K 9/0043; A61K 47/48884; C12N 15/1137; C12N 2320/32; C12N 2310/351

(57) ABSTRACT

The present invention features compositions and methods for targeted delivery of a therapeutic or imaging agent to a site accessible through the nose or mouth that may be difficult to effectively and efficiently treat otherwise (e.g., the middle ear, sinuses, or lung). The therapeutic or imaging agent is deposited onto a magnetic nanoparticle that is drawn through a passage or tissue that leads away from the nose or mouth by a magnetic field applied over the targeted site (e.g., by magnets within the ear canal or surrounding the ear).

### CROSS-REFERENCE TO RELATED APPLICATIONS

This application claims benefit of the filing date of U.S. Provisional Application No. 61/800,654, which was filed Mar. 15, 2013.

### FIELD OF THE INVENTION

The present invention relates to compositions and methods in which magnetized particles, generally on the nanometer scale, are used to deliver therapeutic or and/or imaging agents to anatomical targets that are difficult to access, such as the sinuses and middle ear.

### BACKGROUND

Ear infections are very common among children, producing inflammation in the middle ear known as otitis media. In acute cases, pain is managed with analgesics, and antibiotics may be administered systemically. There is concern about this approach because the analgesics do nothing to combat the underlying infection, and systemic, non-targeted antibiotics can not only produce side effects such as vomiting, but also contribute to antibiotic resistance. Antibiotics and other drugs have been delivered to the middle ear in the form of ear drops. However, this approach is fraught with side effects (Haynes et al., Otolaryngol. Clin. North Am. 40:669-683, 2007; Anderson et al., Int. J. Ped. Otorhinolaryngol. 7:91-95, 1984).

### SUMMARY

The present invention features compositions, methods, and uses for delivering magnetic nanoparticles that are associated with (e.g., conjugated to) therapeutic and/or imaging agents to the body and, in particular, to the lungs or anatomical regions in the head that can be difficult to access. Generally, the magnetized nanoparticle is conjugated to an agent and introduced through the nose (i.e., intranasally) or oral cavity, where it is not swallowed but instead propelled into the nasopharynx for further distribution to the sinuses, middle ear, or lungs. Once the nanoparticles have entered the nose or mouth, perhaps aided by a pressurized delivery system or some other propellant force (e.g., a nasal spray), they are guided to the intended target location with the help of a magnet or magnets that can be externally applied to any given target area in the patient's head, neck, or chest. For example, the nanoparticles could be directed to the middle ear (e.g., through the Eustacian tube) by magnets placed in or over the ears (like ear buds or headphones used to listen to music). Similarly, the nanoparticles could be directed to a sinus cavity by magnets placed on or near the skin over an affected, targeted cavity. While access to the inside of the mouth is generally good, the present compositions and methods may nevertheless be useful in treating conditions affecting the oral cavity or the surrounding tissue (e.g., a cancer of the mouth, tongue, tonsils, uvula, jaw, or lymph nodes in the head or neck). As both therapeutic and detectable agents can be attached to the nanoparticles, the present compositions and methods can also be used in image analysis. Thus, the magnetized nanoparticles can be linked to a fluorescent, luminescent, or otherwise detectable molecule. Where the nanoparticle includes both a detectable molecule and a targeting agent that specifically binds a molecule expressed by, for example, a diseased cell or an invading pathogen, the present methods can also be used to visualize and assess an affected region of a patient's body. Accordingly, the invention encompasses methods of visualizing and assessing a region of a patient's body affected by a disease or condition by administering to the patient a nanoparticle bearing a targeting agent (e.g., an antibody or a biologically active fragment thereof) that specifically binds a molecule (e.g., a cell surface antigen) expressed by the diseased tissue or an invading pathogen. When visualized over time, such assessments can help determine whether a therapeutic approach is having a positive impact on the patient's disease or condition.

In one aspect, the invention features magnetic nanoparticles complexed with an agent that inhibits PDE4B. The nanoparticles can be complexed with the agent via a covalent or non-covalent bond, and the agent can be rolipram, roflumilast, or cilomilast (or a therapeutically active variant, derivative, or prodrug thereof). The agent can also be a nucleic acid that inhibits PDE4B gene expression. In another aspect, the invention features pharmaceutical compositions that include a nanoparticle as described herein (e.g., a magnetic nanoparticle complexed with an agent that inhibits PDE4B, such as rolipram, roflumilast, or cilomilast or an anti-microbial). These compositions can be formulated for intranasal delivery. In another aspect, the invention features methods of topically delivering a composition described herein (e.g., a pharmaceutical composition) to the inner ear of a subject. The methods can include the steps of: administering the composition intranasally to the subject; b) applying magnets to the subject's ears or head; and c) directing the magnetic field toward the subjects middle ear for a time and at a strength sufficient to deliver a therapeutically effective amount of the magnetic nanoparticles to the middle ear. In another aspect, the invention features methods of treating a patient suffering from otitis media. The method can include the steps of: a) administering the pharmaceutical composition of claim 6 to the subject intranasally; b) applying magnets to the subject's ears or head; and c) directing the magnetic field toward the subjects middle ear for a time and at a strength sufficient to deliver a therapeutically effective amount of the magnetic nanoparticles to the middle ear.

Among the advantages of the methods are the ability to deliver an effective treatment with less of a given therapeutic agent. This could reduce the cost of the treatment as well as spare the patient from unpleasant side effects of systemic delivery, such as nausea and vomiting.

### BRIEF DESCRIPTION OF THE DRAWINGS

FIG. 1 is a panel of photomicrographs showing intact, healthy HMEEC cells after exposure to magnetized nanoparticles (conjugated to FITC-A, FITC-B, Gly-A, or Gly-B). S. pneumoniae serves as a positive control and CON represents a negative control.

FIG. 2 is a graph indicating the percent release of LDH after incubation with conjugated magnetized nanoparticles in A549 cells.

### DETAILED DESCRIPTION

The present invention features magnetized nanoparticles that are complexed with therapeutic and/or imaging agents; the nanoparticles can direct the agent to a target region (e.g., the middle ear for the treatment of conditions such as otitis media) when attracted by an externally applied magnetic field.

The Nanoparticles:

We tend to use the term “nanoparticle(s)” because the particles employed must in fact be very small; small enough to pass through bodily tissues and passages without significantly damaging the patient. However, any particle that is small enough to be useful in the present methods can be used, and we use the term “nanoparticle(s)” to refer to all such particles. The nanoparticle may be as small as about 1 nm or it may have a diameter of tens or hundreds of nanometers or more (e.g., having a diameter of about 1 μm to about 500 μm). In some embodiments, the nanoparticle can have a particle size of about 2 to about 20 nm (e.g. about 6 to about 8 nm) or about 50 nm. As noted, the particle size may also be larger, such as from about 100 nm to a few micrometers (e.g., about 150 μm). Other particle sizes may also be selected depending on the particular application. The particles can have any shape, including a generally spherical, cubic, or irregular shape, and the shapes may or may not be substantially uniform. The particle sizes provided above are most usefully referenced when the particle has a generally spherical shape. As particles may have non-spherical shapes and different sizes, the particle size refers to the average size of the particles when used in reference to multiple particles. When a particle has an irregular non-spherical shape, its particle size refers to its effective diameter, which is the diameter of a spherical particle that has the same volume as the non-spherical particle. In cases where the particle has a generally geometrical shape, such as a cuboidal shape, the particle size may refer to a characteristic dimension for that geometrical shape. For example, a cuboidal shape may be characterized by the length of its sides.

One of ordinary skill in the art can determine particle sizes and size distributions using optical or electronic imaging techniques (e.g., TEM) or suitable light scattering techniques such as dynamic light scattering.

The magnetic nanoparticles can include a magnetic core comprising one or more metals such as ferrite (e.g., Fe₃O₄, γ-Fe₂O₃, and CoFe₂O₄). Optionally, the nanoparticles can further include a functionalized coating fashioned from a polymer, hydrogel, polyethylene glycol, glucuronic acid, glycine, or matrix-like materials. The functional coating can serve as a substrate for any number of additional components, including detectable markers (e.g., fluorescent tags such as fluorescein isothiocyanate (FITC) or Rho), targeting agents, and drugs or therapeutic agents. The therapeutic agent can be an anti-inflammatory or anti-microbial agent (e.g., an antibiotic, anti-fungal, or anti-parasitic drug). In some embodiments, the therapeutic agent can be nucleic acid constructs that express CYLD or a biologically active variant thereof (e.g., a variant including the catalytic domain), nucleic acids that inhibit the expression of a negative regulator of CYLD (e.g., PDE4B or JNK2), nucleic acids that modulate the expression of downstream CYLD targets (e.g., Akt, by inhibiting or promoting the expression of the downstream target)

The nanoparticles can be magnetized by any method known in the art. For example, the particles can be fashioned from or may incorporate a metal such as an iron oxide (e.g., Fe₃O₄) or a mixture of different iron oxides (e.g., a mixture of magnetite and maghemite). The magnetic material may be ferromagnetic or superparamagnetic. Strongly magnetic nanoparticles can be manipulated with a weaker magnetic force, as we would expect with Fe₃O₄. However, other, weaker forms of magnetic iron oxides may also be used (e.g., FeO, α-Fe₂O₃, β-Fe₂O₃, γ-Fe₂O₃, and ε-Fe₂O₃). Examples of useful nanoparticles include but are not limited to superparamagnetic iron oxide nanoparticles (SPIOs), ultrasmall superparamagnetic iron oxide nanoparticles (USPIOs having average individual particle diameter of about 10 to 40 nm), monocrystalline iron oxide nanoparticles (MIONs having average particle diameter of about 10 to about 30 nm) or mesoporous silica nanoparticles having attached magnetic particles (MSNs; Yanes and Tamanoi, 2012, Ther. Deliv. 3: 389). A mixture of the nanoparticles conjugated to the same or different therapeutic or detectable agents may be used.

Techniques needed for making magnetic nanoparticles are well known to one of ordinary skill in the art. For example, nanoparticles may be formed by colloidal dispersion formed by wet chemical methods from iron oxides and hydroxides (for example, Alexiou et al., Cancer Res 60:6641, 2000). Magnetic nanoparticles particles may also be obtained from commercial sources. For example, suitable magnetic particles may be obtained from Miltenyi Biotec™, Stemcell Technologies™, Invitrogen™, Pierce™, Ocean Nanotech™ or the like. The raw materials obtained from a commercial source may be further treated to impart one or more characteristics of benefit or added benefit to a patient.

Therapeutic and Imaging Agents:

The nanoparticles may be used in the methods of the invention as either therapeutic or imaging agents or as a combination of the two. In one embodiment, the magnetic nanoparticles are complexed with (e.g., conjugated or electrostatically held to) therapeutic agents. We refer to these complexes as therapeutic nanoparticles. Therapeutic agents in therapeutic nanoparticles include, but are not limited to, small molecule drugs such as antibiotics and enzyme inhibitors; biologics such as peptides, proteins, antibodies, and enzymes including prodrug converting enzymes; plant extracts (such as vinpocetine (referred to as ethyl apovincaminate; Cavinton, Intelectol); nucleic acids including antisense nucleic acids, miRNA, and nucleic acids capable of inducing RNAi. A therapeutic nanoparticle may be conjugated to a single therapeutic agent or plurality of different therapeutic agents (e.g., two or three different therapeutic agents).

In another embodiment, the magnetic nanoparticles are complexed with one or more detectable agents. We refer to these complexes as imaging nanoparticles. Detectable agents include, but are not limited to, fluorescent molecules (fluorochromes or quantum dots), luminescent molecules, dyes, metals, radionuclides, nonradioactive isotopes, or a combination thereof. Optionally, imaging nanoparticles may include an enzyme (such as luciferase) or an enzyme and a substrate, for example, luciferase and luciferin that generate fluorescent or luminescent reaction products. Imaging nanoparticles may optionally further include molecules like antibodies, lectins and receptor ligands to allow binding of the nanoparticles to particular cell types or tissues of interest once the particles have been magnetically localized to the general area. The imaging agents allow the detection of the magnetically localized particles by various methods including, but not limited to, fluorescence- or luminiscence-based imaging, MRI and tomography. Imaging nanoparticles may be used in diagnostics or optionally for directing instruments such as sigmoidoscopes or endoscopes or the like. Imaging nanoparticles may also be used for identifying cell and or tissue types during surgery.

Magnetic nanoparticles may optionally be complexed with both a therapeutic agent and a detectable agents. We refer to these complexes as therapeutic+imaging nanoparticles. Therapeutic+imaging nanoparticles may be targeted to a general area, used in imaging and refocused to a smaller area if necessary by altering magnetic field strengths for precise drug delivery to an affected area or slowly healing area.

In some embodiments, therapeutic and imaging nanoparticles, each complexed with different therapeutic and detectable agents or having different characteristics (such as size or dissolution profiles, described below) can be mixed together.

Means of Complexing and Making Formulations:

The nanoparticles can be conjugated with (a term we use broadly to refer to any specific form of association) a therapeutic or imaging agent by any method known in the art. For example, the nanoparticle and the therapeutic or imaging agent can be linked through a cleavable peptide bond. Following delivery of the particles to the target tissue, the bond may be cleaved by exposure to a relevant protease. In some embodiments, the magnetic nanoparticles are coated with molecules that allow conjugation to an agent. The magnetic nanoparticles may include nonmagnetic material that is added during manufacturing or processing. For example the particles may be coated with suitable matrix including, but not limited to polyvinylpyrrolidone, starch, dextran, polyethylene glycol, calcium alginate, hydroxymethyl cellulose, ethyl cellulose (matrix materials). The matrix agents may be added for ease of making a formulation or conjugating therapeutic and detectable agents.

Conjugation of magnetic nanoparticles with therapeutic or detectable agents may be performed using a variety of different techniques. Chemical groups from the matrix materials may be used as “chemical handles” for covalent cross-linking the therapeutic agents. To allow covalent bond formation, amino, hydroxy, carboxyl and other suitable groups from the matrix agents may be activated and conjugated with appropriate functions group from the drug. Techniques useful for activating these groups are well known to one of ordinary skill in the art. Magnetic nanoparticles having activated chemical groups may be obtained from a commercial vendor. For example, magnetic nanoparticles having activated chemical groups may be obtained from Invitrogen™, Pierce™, or Ocean Nanotech™. The covalent linkages formed may or may not include chemical spacers. The chemical bonds may include but are not limited to peptide, ester, ether, and amide bonds.

Optionally, therapeutic materials are included in the matrix material. Matrix material of the nanoparticles may dissolve immediately and release the included therapeutic or imaging agent. In another embodiment, the matrix materials may be formulated in form of an extended release granule characterized by slow dissolution profile having a magnetic core. This kind of therapeutic agent will allow release of the included therapeutic or imaging agent over an extended time period. Techniques used for making particles for immediate or extended release of therapeutic agents are well known to one of ordinary skill in the art.

Other methods of conjugation include adding an agent to the matrix materials that will bind therapeutic agent with high affinity. For example, the matrix materials may be positively charged and therapeutic agent (such as a nucleic acid) may be a negatively charged. Alternatively or in addition, the therapeutic agent may be an antibody or protein having high affinity to a known ligand and the matrix material may include the ligand.

Matrix materials may also be chosen for improving suspension of the nanoparticles in a given aqueous or non-aqueous carrier suitable for administration. The formulation will depend on site of delivery and contents of magnetic nanoparticles. For oral or nasal administration, the therapeutic or imaging particles may be formulated in form of spray or drops.

In addition to the nanoparticles, the pharmaceutical composition may include additives and/or excipients. The additives and/or excipients may be an combination of isotonic agent(s), buffer(s), surfactant(s), lubricant(s), a preservative(s), a thickening agent(s). Isotonic agents may be, but are not limited to sodium chloride, saccharose, glucose, glycerine, sorbitol, 1,2-propylene glycol. Buffers may include, but are not limited to citrate buffer, phosphate buffer, TRIS buffer, glycine buffer, carbonate buffer. Preservatives may include, but are not limited to edetic acid and its alkali salts, lower alkyl p-hydroxybenzoates, chlorhexidine, phenyl mercury borate, or benzoic acid or a salt, a quaternary ammonium compound, sorbic acid. Lubricants may include but are not limited to magnesium stearate. Thickening agents may include, but not limited to cellulose derivatives, gelatin, pectin, polyvinylpyrrolidone, tragacanth, ethoxose, alginic acid, polyvinyl alcohol and polyacrylic acid.

The Magnetic Field:

The present invention allows delivery of therapeutic/detectable agents to areas difficult to reach by using diagnostic agents conjugated to magnetic nanoparticles using magnetic fields. Upon administration, the nanoparticles are directed to intended areas based on magnetic field generated by a combination of magnets that are externally applied. The resulting magnetic field applied on various locations of body will attract the magnetic nanoparticles to the intended site of delivery. For example, therapeutic agents may be delivered to inner ear by administration in form of nasal spray and wearing magnets in and around ear. Similarly, delivery of therapeutic agent to sinuses may be accomplished by administration of magnetic nanoparticles formulated in a nasal spray and wearing magnets in and around ear, nose and face. Depending on site of delivery, the magnets may be placed inside mouth, inside nose, inside external ear canal, around cheek, around ear, around neck, around chest. The magnets may be designed to be incorporated in ear plugs, ear muffs, hats, neck belts, a magnetic mask that rests on nose and covers part of face under the eyes with elastic belts for attaching to ears, vests, adhesive tapes, and the like. The magnets may be permanent magnets or electromagnets having a power supply and appropriate electronic circuitry to allow adjustment strength of their magnetic fields.

Kits:

The invention also features kits for delivery of therapeutic and/or imaging agents to as inner ear or sinuses. A kit for delivery to inner ear may include (1) a vial and dropper or a nasal spray bottle containing an appropriately formulated therapeutic or imaging agent, (2) magnets and (3) an instruction booklet. The therapeutic or imaging agent is conjugated to a magnetic nanoparticle which is formulated as nasal drops or sprays. Sufficient amounts for multiple administrations may be packaged in a suitable container (such as a vial and a dropper or a nasal spray bottle). Magnets may be supplied in form of ear plugs, ear muffs or a hat. The Instruction booklet provides details of use of the kit including how frequently and how to administer the therapeutic and/or imaging agent; when, how long and how to use the magnets.

A kit for delivery to sinuses may comprise of (1) a vial and dropper or a nasal spray bottle containing an appropriately formulated therapeutic or imaging agent, (2) magnets and (3) an instruction booklet. The therapeutic or imaging agent is conjugated to a magnetic nanoparticle which is formulated as nasal drops or sprays. Sufficient amounts for multiple administrations may be packaged in a suitable container (such as a vial and a dropper or a nasal spray bottle). Magnets may be supplied in form of ear plugs, ear muffs, a magnetic mask that rests on nose and covers part of face under the eyes with elastic belts for attaching to ears, or a hat that covers area of face between eyes and ears. Instruction booklet provides details of use of the kit including how frequently and how to administer the therapeutic and/or imaging agent; when, how long and how to use the magnets.

Other kits can a formulated magnetic nanoparticle that is activated to allow conjugation of drug having a given chemical group(s) (such as hydroxy or amino) or affinity ligand, magnets as described above and instruction booklet. The instruction booklet may contain instructions for conjugation of the drug of the user's choice with the nanoparticles.

Conditions Amenable to Treatment:

Otitis media is a viral or bacterial infection of the ear that is one of the most common childhood infection for which antibiotics are prescribed in the United States. The bacteria responsible for otitis media include Streptococcus pneumoniae, Escherichia coli, Staphylococcus aureus, Haemophilus influenzae, Streptococcus pyogenes, Proteus mirabilis, Klebsiella species and Micrococcus catarrhalis. Studies carried out in developed countries show that by their third birthday, 80% of children will have experienced at least one episode of acute otitis media and 40% will have six or more recurrences by the age of seven years (cited from Monasta et al., 2012, PLoS ONE 7:e36226). The current invention allows delivery of therapeutic agents to inner ear in patients afflicted with this condition. For treatment of otitis media, a drug may be administered in form of nasal spray/drops and magnetic nanoparticles may be directed through the Eustacian tube to middle ear using magnets worn in form of ear plugs, ear muffs and hats. Upon completion of treatment, the magnetic particles are induced to flow to nose by use of special magnetic face mask that concentrates the magnetic field to nose. Nose and sinuses could be washed after delivery to remove the nanoparticles.

The magnetic nanoparticles are also generally useful for treatment of medical conditions associated with inflammation of, or mucus overproduction in, the ears, nose, nasal passages, throat, or lungs. Often these conditions are associated with upregulation of the expression of the deubiquitinase cylindromatosis (CYLD). PDE4B, a cyclic AMP-specific cyclic nucleotide phosphodiesterase, is a negative regulator of CYLD. Specific inhibitors of PDE4B (i.e. an inhibitor that does not significantly inhibit the related protein PDE4D) are potentially useful treatments for such conditions. The patient may be one who does not have cancer.

In one embodiment, an inhibitor of PDE4 is conjugated to a magnetic bead. Exemplary PDE4B inhibitors include rolipram, roflumilast, cilomilast (or a biologically active variant thereof (e.g., a prodrug, derivative, or hydrate thereof) and additional inhibitors described in WO 2007/142929. These inhibitors include a substituted benzene or substituted six-membered heteroaryl rings comprising one or two ring nitrogens, the substitution comprising an ether, thioether, or amine group in which the alkyl group on the ether, thioether, or amine is a haloalkyl group. The haloalkyl group can be a fluoromethyl, difluoromethyl, or trifluoromethyl group. Other inhibitors can be nucleic acids (e.g., a nucleic acid construct) that inhibits PDE4B gene expression. Such nucleic acids are known in the art and include antisense oligonucleotides, microRNAs, and nucleic acids that mediate RNAi (e.g., siRNAs and shRNAs).

The magnetic particle complexed with a PDE4B inhibitor can then be administered intranasally to a patient in need of treatment for otitis media. The patient is given magnets in form of specially designed ear plugs, ear muffs, hat and the like. Upon completion of treatment, the magnetic particles are induced to flow to nose by use of special magnetic face mask that concentrates the magnetic field to nose. The nose and sinuses are then flushed to remove residual particles.

Meniere's disease is an inner ear disorder that affects balance and hearing. The inner ear contains fluid-filled tubes called semicircular canals, or labyrinths. These canals, along with a nerve, help interpret body's position and maintain your balance. In Meniere's disease, this ability is compromised. Sometimes placing the antibiotic gentamicin directly into the middle ear is prescribed to help control vertigo. The current invention will greatly simplify delivery of gentamicin to inner ear. Gentamicin may be administered as a of nasal spray or drops and magnetic nanoparticles may be directed through the Eustacian tube to middle ear using magnets worn in form of ear plugs, ear muffs and hats. At a later time, upon completion of delivery, magnets worn around nose may be used to attract the nanoparticles back to the nose. Similarly, an agent capable of delivering gene therapy for Meniere's disease may be locally delivered using this approach. Upon completion of treatment, the magnetic particles are induced to flow to the nose by use of special magnetic face mask that concentrates the magnetic field to nose. The nose and sinuses can be washed after delivery to remove residual nanoparticles.

Patients suffering from conditions of the lung including, but not limited to, fibrosis, pulmonary tuberculosis, pneumonia, chronic obstructive pulmonary disorder and cystic fibrosis may benefit from the current invention. In these cases, the appropriate therapeutic agent may be administered as nasal spray. Breathing at the time of administration of the spray may propel the nanoparticles to the airways or bronchioles. Thereafter, magnets designed in shape of a vest that covers different parts of chest may direct the nanoparticles to areas of interest.

In another embodiment, the magnetic nanoparticles can be used to treat cystic fibrosis. The therapeutic agent can be a nucleic acid encoding the CFTR gene in a suitable vehicle like a virus or liposome. The CFTR-containing virus or liposome can be conjugated to the magnetic nanoparticles using techniques described above and administered via a nasal spray (in conjunction with breathing in). The particles can be delivered to sites of interest using a magnetic vest or a neck belt. Antibiotics for treating infections associated with cystic fibrosis may also be delivered using the same approach.

Other condition that may benefit from the current invention include tumors in the lymph nodes of the head or neck, particularly those tumor types that are susceptible to steroids or anti-inflammatory treatments. The present invention may also be used for gene therapy.

### EXAMPLES

### Example 1

### Cytotoxicity Analysis of Magnetic Nanoparticles

To examine the cytotoxicity of the magnetic nanoparticles stimulated with or without a magnetic field, HMEEC-1 (human middle ear epithelial-1) or A549 (human lung adenocarcinoma epithelia) cells were incubated for 48 hours to reach 80-90% cell confluency. The nanoparticles were comprised of a magnetic core (CoFe₂O₄), a functionalized coating that was further comprised of glycine (with or without FITC, a fluorescence marker) and polyethylene glycol (with or without 7-amino-methylcoumarin, a fluorescence marker). To this end, the CoFe₂O₄ nanoparticles were prepared via the micelle method. The nanoparticles and polygalacturonic acid (600 mg) were added to 80 mL of a 5 M NaOH solution, sonicated for 5 hours. The coated nanoparticles and separated from the solution using a magnet, washed and then placed in distilled water. Next, the cells were exposed to the magnetized nanoparticles (100 μg/mL, 5×10¹⁴ particles/mL) and divided into four groups with each group receiving either FITC-A, FITC-B, Gly-A or Gly-B while a magnet (2,600 Gauss (2.6 Tesla)) was placed under the cell culture plate for 24 hours to allow the nanoparticles penetrate the cells. S. pneumoniae was used as a positive control.

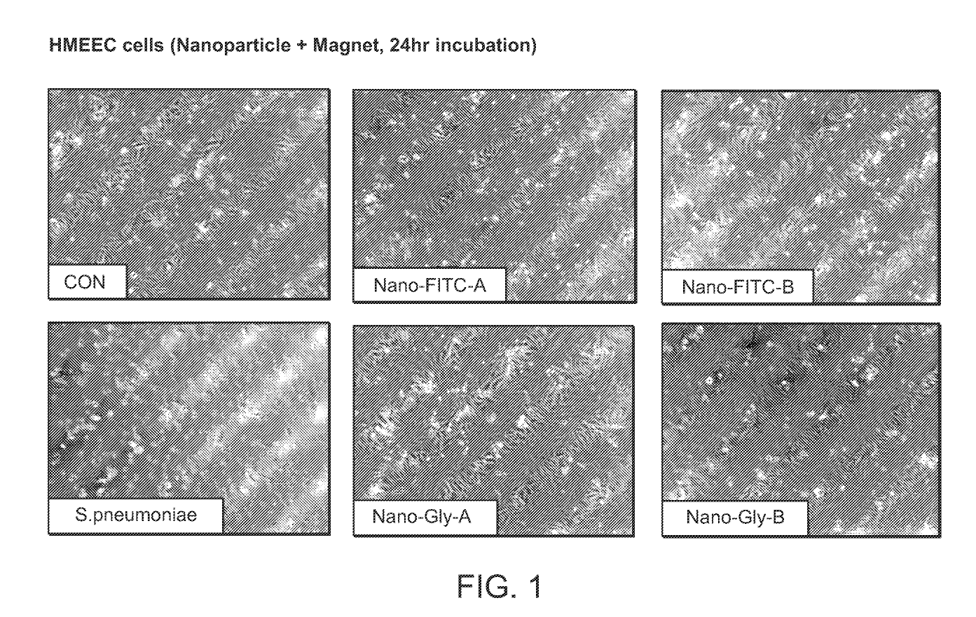

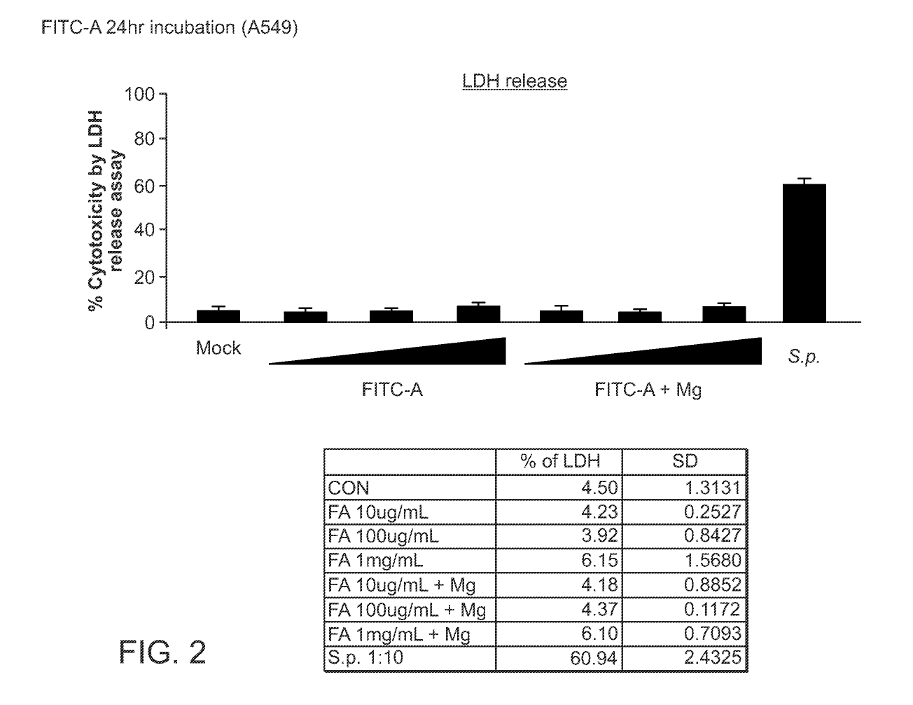

To assess the integrity of the cell membrane, lactate dehydrogenase (LDH) was measured and cell morphology was examined. Both HMEEC-1 (FIG. 1) and A549 cells showed no toxicity at the tested concentration in the presence or absence of the magnetic field. Further, the percent of LDH release was similar in all four groups tested after exposure to the nanoparticles compare to S. pneumoniae in both of the cell lines used. As seen in FIG. 2, the percent release of LDH was markedly lower in cellular groups exposed to the nanoparticles compared to S. pneumoniae. These data suggest that none of the nanoparticles (at a concentration of 100 μg/mL, 5×10¹⁴ particles/mL) led to any significant toxicity in both of the cell lines used.

### Example 2

### In Vitro Cellular Uptake of the Magnetic Nanoparticles

We seeded HMEEC-1 cells in a 24-well cell culture plate at a density of 1.0×10⁵ cells per well and maintained the cells in DMEM supplemented with 10% FBS, penicillin/streptomycin, and BEGM™ (broncial epithelial cell growth medium available from Lonza) SingleQuots® (single-use aliquots typically used for convenience). We incubated the cells for 48 hours until they reached about 80-90% cell confluency. We then exposed the cells to nanoparticles (100 μg/mL, 5×10¹⁴ particles/mL) conjugated to a fluorescent marker (7-amino-4-methylcoumarin) or polyethylene glycol (control) with or without placing a magnet (2,600 Gauss (2.6 Tesla)) under the cell culture plate (the magnet was placed under half of the culture wells and the remainder were unaffected) for 24 hours to allow time for the nanoparticles to penetrate the cells. Similar to the nanoparticles described in Example 1, the nanoparticles used here were comprised of a magnetic core of CoFe₂O₄ and a functionalized coating including polyethylene glycol.

We visualized the cultures under a fluorescence microscope (at 200× magnification) to confirm the appearance of nanoparticle uptake by the HMEEC-1 cells. We observed apparent cellular uptake in the cell cultures exposed to nanoparticles conjugated to fluorescence marker and exposed to an attractant magnet. These data indicate that the magnetized nanoparticles are able to penetrate the cells in response to an external magnet.

What is claimed is:

1. A magnetic nanoparticle complexed with an agent that inhibits PDE4B.

2. The magnetic nanoparticle of claim 1, wherein the nanoparticle is complexed with the agent via a covalent bond.

3. The magnetic nanoparticle of claim 1, wherein the nanoparticle is complexed with the agent via a non-covalent bond.

4. The magnetic nanoparticle of claim 1, wherein the agent is rolipram, roflumilast, or cilomilast.

5. The magnetic nanoparticle of claim 1, wherein the agent is a nucleic acid that inhibits PDE4B gene expression.

6. A pharmaceutical composition comprising a magnetic nanoparticle complexed with an agent that inhibits PDE4B.

7. The pharmaceutical composition of claim 6, wherein the agent is rolipram, roflumilast, or cilomilast.

8. The composition of claim 6, wherein the composition is formulated for intranasal delivery.

9. A method of topically delivering the composition of claim 8 to the inner ear of a subject, the method comprising:
a) administering the composition intranasally to the subject;
b) applying magnets to the subject's ears or head;
c) directing the magnetic field toward the subjects middle ear for a time and at a strength sufficient to deliver a therapeutically effective amount of the magnetic nanoparticles to the middle ear.

10. A method of treating a patient suffering from otitis media, the method comprising:
a) administering the pharmaceutical composition of claim 6 to the subject intranasally;
b) applying magnets to the subject's ears or head;
c) directing the magnetic field toward the subjects middle ear for a time and at a strength sufficient to deliver a therapeutically effective amount of the magnetic nanoparticles to the middle ear.
